(19) United States
(12) Patent Application Publication
(10) Pub. No.: US 2022/0092482 A1
(43) Pub. Date: Mar. 24, 2022

(54) METHOD FOR PREDICTING DIOXIN EMISSION CONCENTRATION

(71) Applicant: Beijing University of Technology, Beijing (CN)

(72) Inventors: Jian TANG, Beijing (CN); Heng XIA, Beijing (CN); Junfei QIAO, Beijing (CN); Zihao GUO, Beijing (CN)

(21) Appl. No.: 17/544,213

(22) Filed: Dec. 7, 2021

Related U.S. Application Data
(63) Continuation of application No. PCT/CN2020/080528, filed on Mar. 21, 2020.

(30) Foreign Application Priority Data
Feb. 10, 2020 (CN) 202010083784.4

(51) Int. Cl. G06N 20/20; G06N 5/02

(52) U.S. Cl. CPC G06N 20/20; G06N 5/02

(57) ABSTRACT

A method for predicting dioxin (DXN) emission concentration based on hybrid integration of random forest (RF) and gradient boosting decision tree (GBDT). A random sampling of a training sample and an input feature is performed on a modeling data with a small sample size and a high-dimensional characteristic to generate a training subset. J RF-based DXN sub-models based on the training subset are established. J×I GBDT-based DXN sub-models are established by performing I iterations on each of the RF-based DXN sub-models. Predicted outputs of the RF-based DXN sub-model and the GBDT-based DXN sub-model are combined by a simple average weighting method to obtain a final output.

### CROSS-REFERENCE TO RELATED APPLICATIONS

This application is a continuation of International Patent Application No. PCT/CN2020/080528, filed on Mar. 21, 2020, which claims the benefit of priority from Chinese Patent Application No. 202010083784.4, filed on Feb. 10, 2020. The content of the aforementioned applications, including any intervening amendments thereto, is incorporated herein by reference in its entirety.

### TECHNICAL FIELD

The present application relates to municipal solid waste incineration, and more particularly to a method for predicting dioxin emission concentration based on hybrid integration of random forest and gradient boosting decision tree.

### BACKGROUND

In China, the rapid development of economy and the continuous expansion of urbanization have led to a dramatic increase in the generation of municipal solid waste (MSW), especially in those economically developed and densely populated districts. More seriously, some cities are suffering from the crisis of garbage siege. Municipal solid waste incineration (MSWI)-based power generation is a typical harmless treatment method for volume reduction and recycling. At present, there are over 300 domestic MSWI power plants, and grate furnace incinerators account for more than ⅔. In view of the particular elemental compositions of Chinese MSW, most of the imported incinerators are operated manually, which leads to the frequent occurrence of “unacclimatization”, as well as the substandard emission of MSWI. Accordingly, how to control the pollution emission of MSWI one the premise of ensuring economic benefits has been the most critical issue. Dioxin (DXN), as a highly toxic persistent organic pollutant produced by MSWI, has strong chemical reactivity and thermal stability, is one of the main reasons for the public thought of “Not-In-My-Back-Yard”.

In the actual operation, the DXN emission concentration is mainly detected at a certain period by a combination of online sampling and offline experimental analysis. However, the foregoing detection is expensive and time consuming; moreover, it is difficult to support the real-time optimization control of MSWI operating parameters to minimize the DXN emission concentration. Therefore, an online prediction of DXN emission concentration is necessary. The complex physical and chemical characteristics of the MSWI process make it difficult to establish a model for precisely predicting the DXN emission concentration. Unfortunately, the online prediction of DXN emission concentration plays a vital role in optimizing the MSWI process. Currently, the online detection of DXN is generally performed by measuring related objects firstly and then performing online prediction through a mapping relationship, which has problems such as expensive equipment, weak adaptability and unsatisfactory prediction precision. Compared to the direct offline analysis and related object detection, the soft sensor method can faster and more economically predict difficult-to-measure parameters, and has been widely used in the industrial field. Regarding the MSWI process, it has been reported to employ a combination of feature selection and neural network to construct a DXN emission concentration prediction model. Due to characteristics of modeling data, such as low sample size, high dimensionality, and collinearity, these methods generally suffer from local minima, overfitting and poor generalization performance.

In view of limitations of traditional single prediction model, a prediction model based on ensemble learning has attracted a lot of attention. Random forest (RF) algorithm has strong capabilities of noise processing and nonlinear data modeling, but is not usually used for nonlinear regression. An online prediction of biomass moisture content in a fluidized bed based on combination of electrostatic sensor arrays and the random forest method has been proposed (Zhang, W. B., Cheng, X. F., Hu, Y. H., Yan, Y., 2019. Online prediction of biomass moisture content in a fluidized bed dryer using. Fuel, 239, 437-445). A soft sensor model based on principal component analysis and RF has been constructed for the online prediction of tensile property of twin-screw extruded polylactide sheet (Mulrennan, K., Donovan, J., Creedon, L., Rogers, I., Lyons, J. G., McAfee, M., 2018. A soft sensor for prediction of mechanical properties of extruded PLA sheet using an instrumented slit die and machine learning algorithms Polymer Testing, 69, 462-469). A literature (Napier, L. F. A., Aldrich, C., 2017. An IsaMill™ Soft Sensor based on Random Forests and Principal Component Analysis. Ifac Papersonline, 50, 1175-1180) provided a self-monitoring RF model for the online estimation of P80 particle size in the mill. In addition to the RF algorithm based on modeling data sampling for parallel integration, the gradient boosting decision tree (GBDT) is another popular machine learning algorithm, whereas, the efficiency and scalability of the GBDT are still unsatisfactory in the case of high feature dimension and large data size. A literature (Sachdeva, S., Bhatia, T., Verma, A. K., 2020. A novel voting ensemble model for spatial prediction of landslides using GIS. International Journal of Remote Sensing, 41, 929-952) provides an ensemble model integrated with logistic regression (LR), GBDT and voting feature interval (VFI) to evaluate a landslide susceptibility.

A literature (Wang, R., Lu, S. L., Li, Q. P., 2019. Multi-criteria comprehensive study on predictive algorithm of hourly heating energy consumption for residential buildings. Sustainable Cities and Society, 49.) utilized the GBDT to predict energy consumption for residential buildings. A literature (Chen, B. B., Lin, R. H., Zou, H., 2018. A Short Term Load Periodic Prediction Model Based on GBDT. 2018 Ieee 18th International Conference on Communication Technology (Icct),1402-1406) established a short-term load periodic prediction model based on the GBDT. A literature (Wang, J. D., Li, P., Ran, R., Che, Y. B., Zhou, Y., 2018. A Short-Term Photovoltaic Power Prediction Model Based on the Gradient Boost Decision Tree. Applied Sciences-Basel 8) provided a photovoltaic power prediction model based on GBDT, in which binary trees are integrated through gradient boosting. It has also been reported to establish a wind power quantile regression model based on an instance-based transfer learning method embedded GBDT (Cai, L., Gu, J., Ma, J. H., Jin, Z. J., 2019. Probabilistic Wind Power Forecasting Approach via Instance-Based Transfer Learning Embedded Gradient Boosting Decision Trees. Energies 12). A literature (Liu, X. L., Tan, W. A., Tang, S., 2019. A Bagging-GBDT ensemble learning model for city air pollutant concentration prediction. 4^{th} International Conference on Advances in Energy Resources and Environment Engineering 237) proposed a Bagging-GBDT ensemble learning prediction model. Nevertheless, the above researches mostly use a single RF of GBDT algorithm for modeling, failing to effectively construct a model with small sample size and high-dimensional characteristics to predict the DXN emission concentration.

### SUMMARY

DXN is a highly toxic pollutant produced by the MSWI. In the current industrial process, the DXN emission concentration is mainly measured by collecting flue gas samples on site and then analyzing the samples in a laboratory, which has problems such as time-consuming operation and high cost. In view of the above-mentioned defects in the prior art, an object of this application is to provide a DXN emission concentration prediction model based on hybrid integration of random forest (RF) and gradient boosting decision tree (GBDT), in which a process control system is employed to acquire a process variable in real time.

Technical solutions of this application are described as follows.

This application provides a method for predicting dioxin emission concentration, comprising:

performing random sampling of a training sample and an input feature on a DXN emission concentration prediction modeling data with a small sample size and a high-dimensional characteristic to generate a training subset;

establishing an RF-based DXN sub-model based on the training subset;

performing an iteration on each of the RF-based DXN sub-model I times to establish a GBDT-based DXN sub-model; and

combining a predicted output of the RF-based DXN sub-model and the GBDT-based DXN sub-model by a simple average weighting method to obtain a final output;

wherein the number of the RF-based DXN sub-model is J; and the number of the GBDT-based DXN sub-model is J×I.

Compared to the prior art, the application has the following beneficial effects. In the method provided herein, a DXN emission concentration prediction model based on hybrid integration of RF and GBDT is established, which has an improved online prediction precision for the DXN emission concentration, and can facilitate the optimization of operation parameters of the MSWI and improve the economic benefit.

### BRIEF DESCRIPTION OF THE DRAWINGS

FIG. 1 depicts a work process of municipal solid waste incineration;

FIG. 2 is a flow chart showing establishment of a model for predicting DXN emission concentration;

FIG. 3 depicts a prediction curve of training data; and

FIG. 4 depicts a prediction curve of test data.

### DETAILED DESCRIPTION OF EMBODIMENTS

### Description of MSWI Process for DXN Generation

MSW is transported by a vehicle to a weighbridge to be weighted and discharged into a garbage pool. After biologically fermented and dehydrated for 3-7 days, the MSW is transferred to a garbage hopper by a grab, fed to an incineration grate through a feeder, and subjected to drying, burning and incineration successively. Combustible components of the MSW after drying are burned in the combustion air delivered by a primary air fan. The ash residue generated by burning falls from an end of the grate to a slag conveyor to be transported to a slag pit, and finally is landfilled at a designated location. A temperature of the flue gas produced in the combustion process should be controlled above 850° C. in a first combustor to ensure a complete decomposition and combustion of harmful gas. When the flue gas passes through a second combustor, air delivered by a secondary air fan generates a turbulence, which ensures that the residence time of the flue gas exceeds 2 s, such that the harmful gas is further decomposed. The flue gas then enters a waste heat boiler and absorbs heat to generate high-temperature steam to drive a turbo-generator set to generate electricity. Subsequently, the flue gas is mixed with lime and activated carbon, and enters a deacidification reactor to undergo a neutralization reaction to allow the DXN and heavy metals therein to be adsorbed. Then, a flue gas particle, neutralization reactant and activated carbon are removed in a bag filter. Part of the gas and ash mixture is mixed with water in a mixer and then transported into the deacidification reactor for repeated treatment. Fly ash produced in the deacidification reactor and the bag filter enters a fly ash tank and is needed to be transported to experience further processing. The final gas is emitted to the atmosphere by an induced draft fan through a stack, which includes soot, CO, NOx, SO2, HC1, HF, Hg, Cd, DXN and so on.

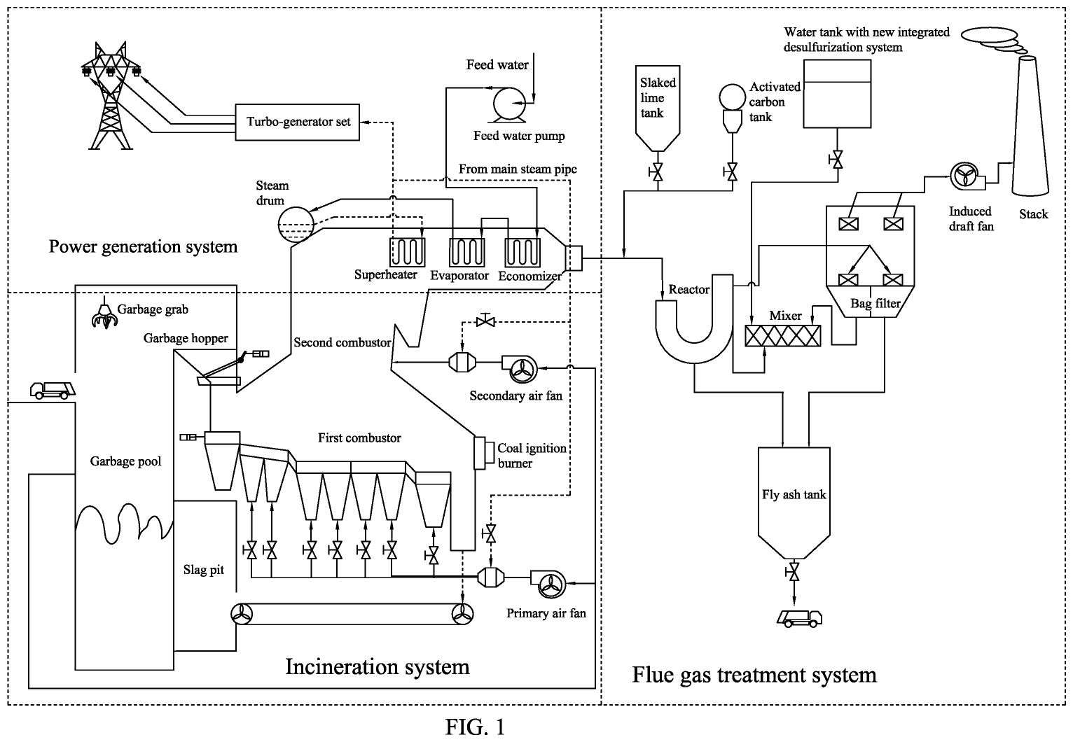

As shown in FIG. 1, the MSWI is mainly used to convert the MSW into slag, fly ash, flue gas and heat, where the slag, fly ash and flue gas are related to the DXN emission. The furnace residue has a large generation amount but a relatively low DXN concentration, while the fly ash has a lower generation amount, but a larger DXN concentration. The DXN in flue gas is mainly derived from the incomplete combustion and the synthesis reaction. At present, a detection of DXN is mainly performed monthly or quarterly by enterprises and environmental protection departments off line, which is expensive and time consuming. Indicatively, the DXN emission concentration prediction modeling data has problems such as few true value sample and high dimensionality of process variable, as well as other objective problems such as unknown DXN content in MSW and complicated and unclear DXN generation and absorption mechanism. Therefore, establishing a DXN emission concentration prediction model based on a soft sensor method is necessary.

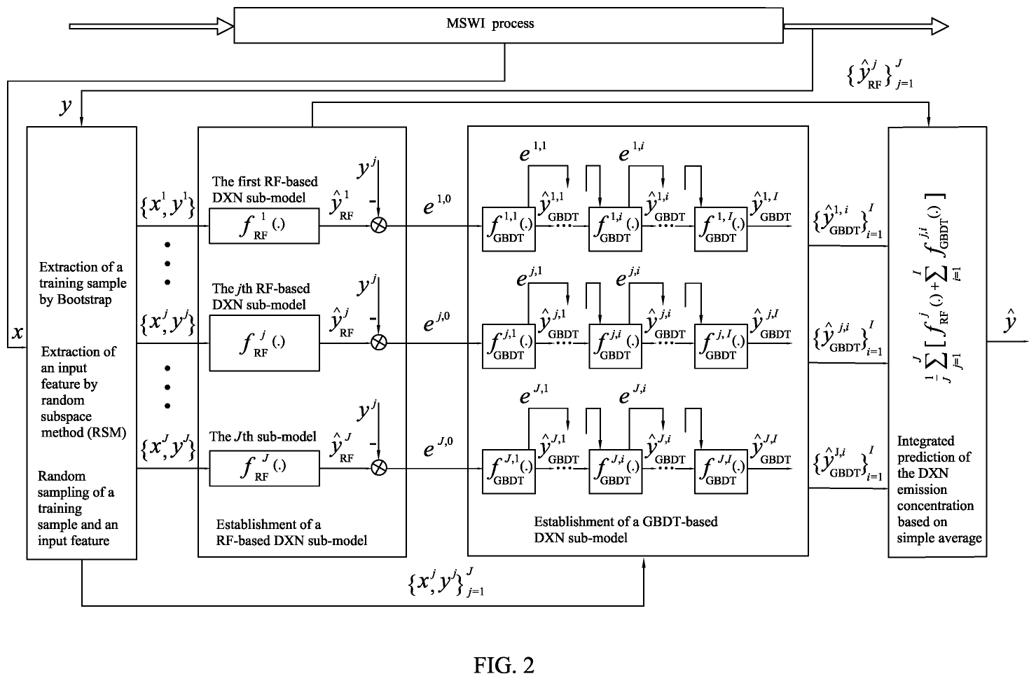

As shown in FIG. 2, a modeling strategy of the DXN emission concentration prediction model based on hybrid integration of random forest (RF) and gradient boosting decision tree (GBDT) (EnRFGBDT) includes a training sample and input feature random sampling module, a RF-based DXN sub-model establishing module, a GBDT-based DXN sub-model establishing module and a a simple average-based DXN integrated prediction module.

In FIG. 2, X={xₙ}ₙ₋₁^{N}∉R^{N×M}, which represents an input data {x|x₁, . . . , xₘ, . . . x_{M} } consisting of a process variable (input feature) of a municipal solid waste incineration (MSWI) process acquired by a process control system while collecting a DXN test sample. The process variable includes furnace temperature, activated carbon injection amount, stack emission gas concentration, grate speed, primary air flow and secondary air flow. N is the number of training samples. M is the number of the process variable. and y={yₙ}ₙ₌₁^{N}∉R^{N×1}, which represents an output data consisting of the DXN emission concentration at an end of the MSWI process, where the end of the MSWI process is a stack emission end, and the DXN emission concentration is obtained by online collection and offline analysis. {X, y} is a training sample set consisting of the input data and the output data. {X^{j}, y^{j}} is a training subset the jth obtained by random sampling from {X, y}, and {X^{j}, y^{j}}ⱼ₌₁^{J} is all of the training subsets. J is the number of the training subset as well as the number of the RF-based DXN sub-models. ŷ_{RF}^{j} is a predicted value of the DXN emission concentration of a jth RF-based DXN sub-model f_{RF}^{j}(⋅). {ŷ_{RF}^{j}}ⱼ₌₁^{J} is is a predicted output of all of the RF-based DXN sub-models. e^{j,0} is a predicted error between the predicted output Sr^{j}_{RF} of the DXN emission concentration of the jth RF-based DXN sub-model and a measured value y^{j} of DXN emission concentration of the jth RF-based DXN sub-model. e^{j,1} is an output residual between a predicted output ŷ_{GBDT}^{J,1} of a jth training subset of the first GBDT-based DXN sub-model and the e^{j,0} which is as a true value of output data of the jth training subset of the first GBDT-based DXN sub-model. e^{j,i} is an output residual between a predicted output of a jth training subset of a ith GBDT-based DXN sub-model f_{GDBT}^{j,i}(⋅) and e^{j,i-1} which is as a true value of input data of the jth training subset of the ith GBDT-based DXN sub-model. {ŷ_{GBDT}^{j,i}}ᵢ₌₁^{I} is a predicted output of a jth training subset of all of the GBDT-based DXN sub-models, where I is the number of the GBDT-based DXN sub-model of one training subset, also is the number of iterations of the training subset. ŷ is a predicted output of a DXN emission concentration prediction model based on hybrid integration of RF and GBDT.

All of sub-models of the DXN emission concentration prediction model based on hybrid integration of EnRFGBDT herein are established by maximize growth classification and regression trees (CART). The training subset of the RF-based DXN sub-model and the input feature of the RF-based DXN sub-model are generated by random sampling, where the number of features of the RF-based DXN sub-model is much smaller than that of an initial modeling data, therefore a correlation between the CART is reduced, and a robustness of an outlier and a noisy data are improved. Multiple GBDT-based DXN sub-models in series further improve a prediction precision of the CART. As a consequence, the DXN emission concentration prediction model in “parallel+series” is established. Different modules are performed as follows.

(1) A random sampling with replacement N time to the training sample set {X∉R^{N×M}, y∉R^{N×1}} and a random selection of a fixed number of input features from the training sample set are performed by the training sample and input feature random sampling module to generate the training subset {X^{j}, y^{j}}ⱼ₋₁^{J}.

(2) A RF-based DXN sub-model {f_{RF}^{j}(⋅)}ⱼ₌₁^{J} is established by the random forest (RF)-based DXN sub-model establishing module. A predicted value {ŷ^{j}}ⱼ₌₁^{J} of the DXN emission concentration is subtracted from a measured value {y^{j}}ⱼ₌₁^{J} of the DXN emission concentration to obtain a prediction error {e^{j,0}}ⱼ₌₁^{J}.

(3) I iterations are performed on each of a new training subset {X^{j}, e^{j,0}}ⱼ₌₁^{J} to build I×J GBDT-based DXN sub-models {{f_{GBDT}^{j,i}(⋅)}ᵢ₌₁^{I}}ⱼ₌₁^{J} by the GBDT-based DXN sub-model establishing module, where the new training subset is formed by the prediction error {e^{j,0}}ⱼ₋₁^{J} as an output data true value and an input data of a training subset {X^{J}}ⱼ₋₁^{J}.

(4) The RF-based DXN sub-model {ŷ_{RF}^{J}}ⱼ₋₁^{J} and the GBDT-based sub-model {{f_{GBDT}^{j,i}(⋅)}ⱼ₋₁^{I}}ⱼ₋₁^{J} are subjected to simple averaging by the simple average-based DXN integrated prediction module to establish the DXN emission concentration prediction model f_{DXN}(⋅).

Accordingly, steps of modeling the method herein is as follows.

(1) A random sampling with replacement and a random selection of a fixed number of input features are performed on the process variable of the MSWI process to generate J training subsets.

(2) J RF-based DXN sub-models {f_{RF}^{j}(⋅)}ⱼ₋₁^{J} are established.

(3) I iterations are performed to build I×J GBDT-based DXN sub-models {{f_{GBDT}^{j,i}(⋅)}ⱼ₋₁^{I}}ⱼ₋₁^{J} where the prediction error {e^{j,0}}ⱼ₋₁^{J} of the {f_{RF}^{j}(⋅)}ⱼ₋₁^{J} is used as the output data true value.

(4) The RF-based DXN sub-model and the GBDT-based sub-model are subjected to simple averaging to establish the DXN emission concentration prediction model.

### Description of Work Process Of Training Sample and Input Feature Random Sampling Module

The process variable of the MSWI process is processed by a Bootstrap method and a random subspace method (RSM).

The training subset is extracted by the Bootstrap method, where the number of samples in the training subset is the same with the number of samples of the training sample set.

Then the RSM is introduced to randomly select some features to generate J training subsets including N training samples and M^{j} input features.

Generation of the training subset is expressed as follows:

$$\begin{matrix}X = {\{ {x}_{n} \}}_{n = 1}^{N} ∈ {R}^{N \times M} \\ y = {\{ {y}_{n} \}}_{n = 1}^{N} \\ J\end{matrix} \} ⇒ ⇒ \{ \begin{matrix}\{ {X}^{1} , {y}^{1} \} = {\{ {( {x}^{1 , {M}^{1}} , {y}^{1} )}_{n} \}}_{n = 1}^{N} \\ … \\ \{ {X}^{j} , {y}^{j} \} = {\{ {( {x}^{j , {M}^{j}} , {y}^{j} )}_{n} \}}_{n = 1}^{N} \\ … \\ \{ {X}^{J} , {y}^{J} \} = {\{ {( {x}^{J , {M}^{J}} , {y}^{J} )}_{n} \}}_{n = 1}^{N}\end{matrix} \qquad ( 1 )$$

where {X^{j},y^{j}} is a jth training subset; (x^{j,M}^{^{j}}, y^{j})ₙ is a nth input-output sample pair of the jth training subset; m=1, . . . , M^{j}, M^{j} is the number of input features in the jth training subset; and M^{j}<<M.

### Description of Work Process of Work Process of RF-Based DXN Sub-Model Establishing Module

The RF-based DXN sub-model establishing module is operated through the following steps with the jth training subset {(x^{j,M}^{^{j}}, y^{j})ₙ}ₙ₌₁^{N} as an example.

A duplicate sample is removed from the jth training subset {(x^{j,M}^{^{j}}, y^{j})ₙ}ₙ₌₁^{N} caused by random sampling the duplicate sample is marked as

$${\{ {( {x}^{j , {M}^{j}} , {y}^{j} )}_{{n}_{sel}} \}}_{{n}_{sel} = 1}^{{N}_{sel}} .$$

A mth input feature x^{j,m} is taken as a splitting variable, and a value xₙ_{ₛₑₗ}^{j,m} corresponding to the nₛₑₗth sample is taken as a splitting point, such that an input feature space is split into two areas R₁ and R₂ respectively.

$$\{ \begin{matrix}{R}_{1} ( m , {x}_{{n}_{sel}}^{j , m} ) = \{ {x}^{j , {M}^{j}} \vert {x}^{j , m} \leq {x}_{{n}_{sel}}^{j , m} \} \\ {R}_{2} ( m , {x}_{{n}_{sel}}^{j , m} ) = \{ {x}^{j , {M}^{j}} \vert {x}^{j , m} > {x}_{{n}_{sel}}^{j , m} \}\end{matrix} \qquad ( 2 )$$

A number of an optimal splitting variable and a value of the splitting point are found based on the following criterion by traversing all input features:

$${min}_{m , {x}_{{n}_{sel}}^{j , m}} {[ {min}_{{C}_{1}} {\sum }_{{x}^{j , m} ∈ {R}_{1} ( m , {x}_{{n}_{sel}}^{j , m} )} ( {y}_{1}^{j} - {C}_{1} ) )}^{2} + {min}_{{C}_{2}} {\sum }_{{x}^{j , m} ∈ {R}_{2} ( m , {x}_{{n}_{sel}}^{j , m} )} {( {y}_{2}^{j} - {C}_{2} )}^{2} ] \qquad ( 3 )$$

where y₁^{j} and y₂^{j} are a measured value of DXN emission concentration of the jth training subset in the R₁ and the R₂, respectively; and C₁ and C₂ are a mean value of a measured value of DXN emission concentration in the R₁ and the R₂, respectively.

The above processes are repeated respectively for R₁ and R₂ until the number of training samples in a leaf node is less than a preset threshold ∂_{RF} to split the input feature space into K areas. The K areas are marked as R₁, . . . , Rₖ, . . . , R_{K}, respectively, where K indicates the number of the leaf node of the CART.

The RF-based DXN sub-model established by the CART is expressed as follows

$$\begin{matrix}{\hat{y}}_{RF}^{j} = {f}_{RF}^{j} ( \cdot ) = {\sum }_{k = 1}^{K} {c}_{RF}^{k} I ( {x}^{j , {M}^{j}} ∈ {R}_{k} ) & ( 4 ) \\ {c}_{RF}^{k} = \frac{1}{{N}_{{R}_{k}}} {\sum }_{{n}_{{R}_{k}} = 1}^{{N}_{{R}_{k}}} {y}_{{n}_{{R}_{k}}}^{j} , {N}_{{R}_{k}} \leq {θ}_{RF} & ( 5 )\end{matrix}$$

where N_{R}_{ₖ} is the number of the training sample in the area Rₖ;

$${y}_{{n}_{{R}_{k}}}^{j}$$

is a n_{R}_{ₖ}th measured value in the area Rₖ of the jth training subset; I(⋅) is an indicator function; and when x^{j,M}^{^{j}}∉Rₖ, I(⋅)=1, otherwise I(⋅)=0 .

A prediction error of the RF-based DXN sub-model established based on the jth training subset {(x^{j,M}^{^{j}}, y^{j})ₙ}ₙ₌₁^{N} is expressed as follows:

$${e}^{j , 0} = {y}^{j} - {\hat{y}}_{RF}^{j} = {\{ {( {e}^{j , 0} )}_{n} \}}_{n = 1}^{N} \qquad ( 6 )$$

where (e^{j,0})ₙ is a predicted error of DXN emission concentration based on a nth training sample.

The above processes are repeated to obtain J RF-based DXN sub-models {f_{RF}^{j}(⋅)}ⱼ₌₁^{J} established by the CART. A predicted output {ŷ_{RF}^{j}}ⱼ₌₁^{J} of the J RF-based DXN sub-models respectively is subtracted from a measured value {y^{j}}ⱼ₌₁ to obtain the prediction error {e^{j,0}}ⱼ₌₁^{J}.

### Description of Work Process of Work Process of the GBDT-Based DXN Sub-Model Establishing Module

Multiple weak learner models “in series” are established, where an input data of a training subset of the multiple weak learner models is unchanged. A true value of output data of a training subset of a first GBDT-based DXN sub-model is an error between the predicted output of the RF-based DXN sub-model and the measured value. And a true value of output data of a training subset of other GBDT-based DXN sub-models is a prediction error of the GBDT-based DXN sub-model iterated in a previous iteration.

Establishment of a jth GBDT-based DXN sub-model is taken as an example. I GBDT-based DXN sub-model are supposed to be established by the CART.

A first GBDT-based DXN sub-model is established:

$${\hat{y}}_{GBDT}^{j , 1} = {f}_{GBDT}^{j , 1} ( {\{ {( {x}^{j , {M}^{j}} )}_{n} \}}_{n = 1}^{N} , {\{ {( {e}^{j , 0} )}_{n} \}}_{n = 1}^{N} ) ) \qquad ( 7 )$$

where ŷ_{GBDT}^{j,1} is the predicted output of the first GBDT-based DXN sub-model.

A loss function of the first GBDT-based DXN sub-model is defined as follows:

$${L}_{GBDT} ( {y}^{j} , {\hat{y}}_{GBDT}^{j , 1} ) = \frac{1}{2} {\sum }_{n = 1}^{N} {( {( {e}^{j , 0} )}_{n} - {( {\hat{y}}_{GBDT}^{j , 1} )}_{n} )}^{2} \qquad ( 8 )$$

where (ŷ_{GBDT}^{j,1})ₙ is a predicted value of a nth sample in a jth training subset.

An output residual e^{j,1} of the first GBDT-based DXN sub-model f_{GBDT}^{j,1}(⋅) is calculated, which is expressed as follows:

$${e}^{j , 1} = {e}^{j , 0} - {f}_{GBDT}^{j , 1} ( \cdot ) = {y}^{i} - {f}_{RF}^{j} ( \cdot ) - {f}_{GBDT}^{j , 1} ( \cdot ) = {y}^{j} - {\hat{y}}_{RF}^{j} - {\hat{y}}_{GBDT}^{j , 1} \qquad ( 9 )$$

The e^{j,1} is taken as a true value of output data of a training subset of a second GBDT-based DXN sub-model f_{GBDT}^{j,2}(⋅). The second GBDT-based DXN sub-model is expressed as follows:

$${\hat{y}}_{GBDT}^{j , 2} = {f}_{GBDT}^{j , 2} ( {\{ {( {x}^{j , {M}^{j}} )}_{n} \}}_{n = 1}^{N} , {\{ {( {e}^{j , 1} )}_{n} \}}_{n = 1}^{N} ) ) \qquad ( 10 )$$

where (e^{j,1})ₙ is a predicted error of the first GBDT-based DXN sub-model of the nth sample.

The above processes are repeated. A ith (i≤I) GBDT-based DXN sub model is marked as f_{GBDT}^{j,i}(⋅) where an output residual of the ith GBDT-based DXN sub-model is expressed as follows:

$${e}^{j , i} = {y}^{j} - {f}_{RF}^{j} ( \cdot ) - {f}_{GBDT}^{j , 1} ( \cdot ) - , ⋯ , - {f}_{GBDT}^{j , i} ( \cdot ) = {y}^{j} - {\hat{y}}_{RF}^{j} - {\hat{y}}_{GBDT}^{j , 1} - , ⋯ , - {\hat{y}}_{GBDT}^{j , i} . \qquad ( 11 )$$

After I−1 iterations, a true value of output data of a training subset of a Ith GBDT-based DXN sub-model is expressed as follows:

$${e}^{j , I - 1} = {y}^{j} - {\hat{y}}_{RF}^{j} - {\hat{y}}_{GBDT}^{j , 1} - , ⋯ , - {\hat{y}}_{GBDT}^{j , i} , ⋯ , - {\hat{y}}_{GBDT}^{j , I - 1} \qquad ( 12 )$$

where ŷ_{GBDT}^{j,I-1} is a predicted output of a (I−1)th GBDT-based DXN sub-model.

The Ith GBDT-based DXN sub-model is expressed as follows:

$${\hat{y}}_{GBDT}^{j , I} = {f}_{GBDT}^{j , I} ( {\{ {( {x}^{j , {M}^{j}} )}_{n} \}}_{n = 1}^{N} , {\{ {( {e}^{j , I - 1} )}_{n} \}}_{n = 1}^{N} ) ) \qquad ( 13 )$$

where (e^{j,I-1})ₙ is a predicted error of the (I−1)th GBDT-based DXN sub-model for the nth sample.

As a consequence, the I GBDT-based DXN sub-models based on the jth training subset are expressed as {f_{GBDT}^{j,i}(⋅)}ᵢ₌₁^{I}, and an output of the I GBDT-based DXN sub-models is expressed as {ŷ_{GBDT}^{j,i}}ᵢ₌₁^{I},

### Description of Work Process of Work Process of Simple Average-Based DXN Integrated Prediction Module

J RF-based DXN sub-models established in parallel are indicate as {f_{RF}^{j}(⋅)}ⱼ₌₁^{J}. J×I GBDT-based DXN sub-models established in series and parallel simultaneously are indicated as

$${\{ {( {f}_{GBDT}^{j , i} ( \cdot ) )}_{i = 1}^{I} \}}_{j = 1}^{J} .$$

For the jth training subset, one RF-based DXN sub-model and I GBDT-based DXN sub-models are established in parallel. A sum of a predicted output of the one RF-based DXN sub-model and the I GBDT-based DXN sub-models are taken as a total output of the jth training subset, which is expressed as follows:

$${\hat{y}}^{j} = {\hat{y}}_{RF}^{j} + {\hat{y}}_{GBDT}^{j , 1} + , ⋯ , + {\hat{y}}_{GBDT}^{j , i} , ⋯ , + {\hat{y}}_{GBDT}^{j , I - 1} = {\hat{y}}_{RF}^{j} + {\sum }_{i = 1}^{I} {\hat{y}}_{GBDT}^{j , i} = {f}_{RF}^{j} ( \cdot ) + {\sum }_{i = 1}^{I} {f}_{GBDT}^{j , i} ( \cdot ) . \qquad ( 14 )$$

Since the J training subsets are parallel, the one RF-based DXN sub-model is combined with the I pieces of GBDT-based DXN sub-model through simple average weighting method, where the prediction model f_{DXN} (⋅) is expressed as follows:

$$\hat{y} = {f}_{DXN} ( \cdot ) = \frac{1}{J} {\sum }_{j = 1}^{J} {\hat{y}}^{j} = \frac{1}{J} {\sum }_{j = 1}^{J} ( {f}_{RF}^{j} ( \cdot ) + {\sum }_{i = 1}^{I} {f}_{GBDT}^{j , i} ( \cdot ) ) . \qquad ( 15 )$$

### Description of Work Process of Prediction of DXN Emission Concentration Based on EnRFGBDT Method

The DXN emission concentration prediction model is established based on the training sample and input feature random sampling module, the RF-based DXN sub-model establishing module, the GBDT-based DXN sub-model establishing module and the simple average-based DXN integrated prediction module.

The process variable of the MSWI process, including furnace temperature, activated carbon injection amount, stack emission gas concentration, grate speed, primary air flow and secondary air flow, is taken as an input {x|x₁, . . . , xₘ, . . . x_{M}} of the DXN emission concentration prediction model. The input is calculated successively by the RF-based DXN sub-model establishing module, the GBDT-based DXN sub-model establishing module and the simple average-based DXN integrated prediction module, and a current DXN emission concentration value is as a DXN emission concentration predicted value of the MSWI process.

### Experimental Verification

### Modeling Data

The modeling data herein is the inspection data of the incinerator 1# and incinerator 2# of a MSWI power plant in Beijing in the past 6 years, including the process variable as the input data and the measured value of the DXN emission concentration as the output data. The process variable is obtained from 53 power generation systems, 115 public electrical systems, 14 waste heat boiler systems, 79 incineration systems, 20 flue gas treatment systems and 6 terminal detection systems. The DXN emission concentration is obtained by online collection and offline analysis, and an unit of the DXN emission concentration is ng/Nm³. ⅔ of the 67 samples (45 samples) are used as training data and ⅓ (22 samples) are used as testing data.

### Modeling Experiment

A square error is taken as the loss function both in RF method and GBDT method. The number of the training sample is 45. A range of the number of input feature is [10,20,30,40,50,60,70,80,90,100]. A range of the iteration time of the GBDT is [1,2,3,4,5,6,7,8,9]. The minimum number of the training sample included in the leaf node of the CART is 3. An out-of-bag data (OOB) sampled by the Bootstrap algorithm is configured to perform model testing, with a root-mean-square error (RMSE) as an evaluation index.

For a DXN emission concentration prediction model based on RF, a relationship between the number of input feature and an OOB error is shown in the Table 1, where the number of the CART is 5 (expressed as an average of 50 experiments).

TABLE 1
OOB error under different number
of input features

| The number of | The number of | |
|---|---|---|
| CART | input feature | OOB error |
| 5 | 5 | 1.254442 |
| 5 | 10 | 1.088729 |
| 5 | 15 | 1.071183 |
| 5 | 17 | 1.083559 |
| 5 | 20 | 1.186283 |
| 5 | 25 | 1.140331 |
| 5 | 30 | 1.254986 |

As shown in Table 1, the OOB error is minimum in the case of 15 input features. A relationship between the CART in the DXN emission concentration prediction model based on RF and the OOB error is shown in Table 2, where the number of the input features is unchanged. The experimental result is an average of 50 experiments.

TABLE 2
OOB error with different number of CART

| The number | The number | |
|---|---|---|
| of input feature | of CART | OOB error |
| 15 | 10 | 1.1185 |
| 15 | 20 | 1.0924 |
| 15 | 30 | 1.08139 |
| 15 | 40 | 1.0806 |
| 15 | 50 | 1.0972 |
| 15 | 60 | 1.1153 |
| 15 | 70 | 1.1128 |
| 15 | 80 | 1.1281 |
| 15 | 90 | 1.1248 |
| 15 | 100 | 1.1280 |

As shown in Table 2, the 00B error of the DXN emission concentration prediction model based on RF is minimum in the case of 40 CART, which is slightly higher than the minimum in the Table 1. Therefore, an optimization is required both in the number of the CART and the number of the input features to obtain a better prediction performance.

For a DXN emission concentration prediction model based on GBDT, a relationship between a loss function of square error and an iteration time is shown as Table 3.

TABLE 3
Relationship between loss function of square error and iteration time of
DXN emission concentration prediction model based on GBDT

| Iteration | | |
|---|---|---|
| time | The number of CART | Value of loss function |
| 0 | 1 | 44.0000 |
| 1 | 2 | 3.6268 |
| 2 | 3 | 0.6641 |
| 3 | 4 | 0.1888 |
| 4 | 5 | 0.05339 |
| 5 | 6 | 0.02496 |
| 6 | 7 | 0.008141 |
| 7 | 8 | 0.003389 |
| 8 | 9 | 0.002034 |
| 9 | 10 | 0.001011 |

As shown in Table 3, the value of loss function is gradually decreased as the iteration time increases. A decreasing of the square error is slowed when the iteration time reaches 5. Accordingly, an appropriate iteration time is necessary for reducing a computing consumption.

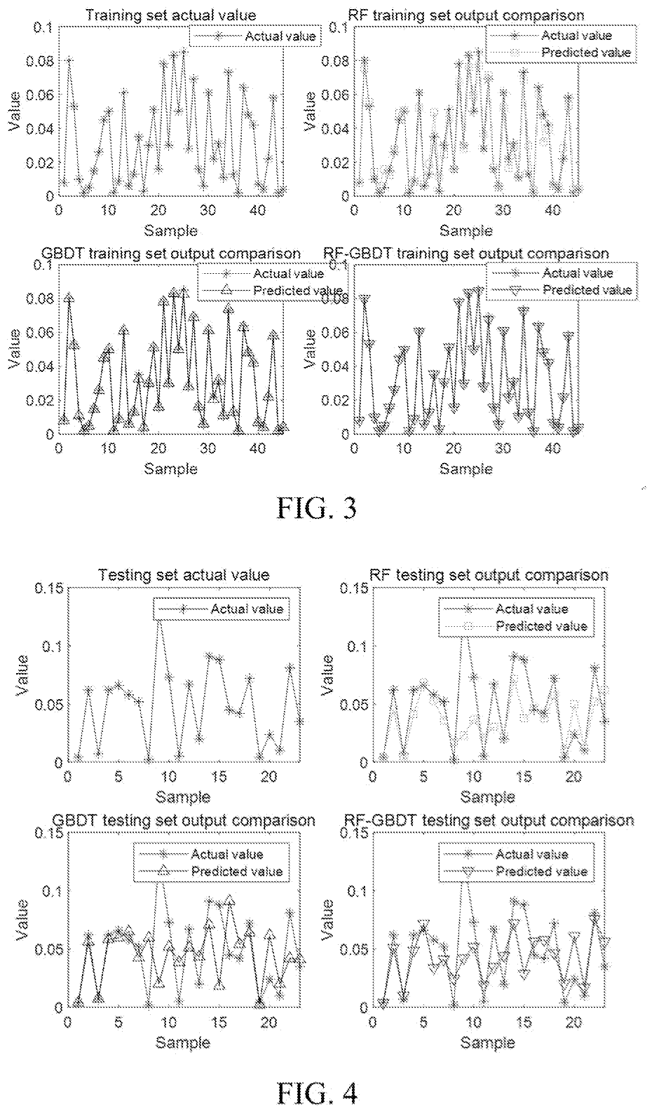

Therefore, a preferable parameter herein is as follows: the number of the input feature is 10; the number of the CART is 5; and the number of the GBDT-based DXN sub-model (iteration time) is 5. Statistical results of training set and testing set based on different method is shown in Table 4. FIGS. 3 and 4 shows prediction curves of DXN emission concentration prediction models constructed respectively based on RF, GBDT and a combination thereof.

TABLE 4
Statistical results of DXN emission concentration prediction
models constructed respectively based on RF,
GBDT and a combination thereof

| | Training set | Test set |
|---|---|---|
| Method | RMSE | RMSE |
| RF | 0.34060 | 0.03019 |
| GBDT | 0.02355 | 0.03529 |
| EnRFGBDT | 0.01478 | 0.02844 |

As shown in Table 4, FIG. 3 and FIG. 4, (1) in the testing set, the DXN emission concentration prediction models based on GBDT has the maximum predicted error which is 0.03529. A main reason is that the GBDT has taken all of process variables as input features of the DXN emission concentration prediction models based on GBDT, while the other two methods have reduced the number of the input feature. Indicatively, a selection of feature is necessary for high-dimensional process variable. (2) When the number of the CART is 5 and the number of the input features is 15, in the training set, the DXN emission concentration prediction models based on RF has the maximum predicted error which is 0.34060. In the testing set, the RMSE of the prediction model based on RF, which is 0.030199, is less than that of the prediction model based on GBDT, which is 0.035291. Indicatively, the generalization ability of RF method is stronger than that of the GBDT method. (3) The EnRFGBDT method herein has the best prediction performance both in training set and testing set, which indicates that the dimension of the input feature is reduced and the generalization ability of the prediction model by EnRFGBDT is improved simultaneously.

Based on the process variable of MSWI process, for a technical problem of detection of DXN emission concentration in real-time, a DXN emission concentration prediction model based on hybrid integration of RF and GBDT is provided. The prediction model herein has following novelty: the first DXN sub-model is established based on RF, and other multiple DXN sub-models are established based on GBDT. The dimension is reduced and the predicted error of the prediction model herein is reduced, simultaneously. Result of simulation experiments based on real data of the MSWI process indicates that the method for predicting herein has an outstanding prediction performance compared to prediction model merely based on RF or GBDT.

What is claimed is:

1. A method for predicting dioxin (DXN) emission concentration, comprising:
(S1) performing, by a training sample and input feature random sampling module, a random sampling with replacement on a training sample set {X∉R^{N×M}, y∉R^{N×1}}N times and a random selection of a fixed number of input features from the training sample set to generate a training subset wherein X={xₙ}ₙ₌₁^{N}∉R^{N×M}, which represents an input data {x|x₁, . . . , xₘ, . . . x_{M}} consisting of a process variable of a municipal solid waste incineration (MSWI) process acquired by a process control system while collecting a DXN test sample; the process variable comprises furnace temperature, activated carbon injection amount, stack emission gas concentration, grate speed, primary air flow and secondary air flow; N is the number of training samples; M is the number of the process variable; and y={yₙ}ₙ₌₁^{N}∉R^{N×1}, which represents an output data consisting of the DXN emission concentration at an end of the MSWI process, wherein the end of the MSWI process is a stack emission end, and the DXN emission concentration is obtained by online collection and offline analysis;
(S2) establishing, by a random forest (RF)-based DXN sub-model establishing module, a RF-based DXN sub-model {f_{RF}^{j}(⋅)}ⱼ₌₁^{J} by utilizing the training subset {X^{j}, y^{j}}ⱼ₌₁^{J}; and subtracting a predicted value {ŷ^{j}}ⱼ₌₁^{J} of the DXN emission concentration from a measured value {y^{j}}ⱼ₌₁^{J} of the DXN emission concentration to obtain a prediction error {e^{j,0}}ⱼ₌₁^{J};
(S3) performing, by a gradient boosting decision tree (GBDT)-based DXN sub-model establishing module, iteration I times on each of a new training subset {X^{j}, e^{j,0}}ⱼ₌₁^{J} to build I×J GBDT-based DXN sub-models {{f_{GBDT}^{j,i}(⋅)}ⱼ₌₁^{J}}ⱼ₌₁^{J}; wherein the new training subset is formed by the prediction error {e^{j,0}}ⱼ₌₁^{J} as an output data true value and an input data of a training subset {X^{j}}ⱼ₌₁^{J};
(S4) subjecting, by a simple average-based DXN integrated prediction module, the RF-based DXN sub-model {ŷ_{RF}^{j}}ⱼ₌₁^{J} and the GBDT-based sub-model {{f_{GBDT}^{j,i}(⋅)}ⱼ₌₁^{J}}ⱼ₌₁^{J} to simple averaging to establish a DXN emission concentration prediction model f_{DXN}(⋅); and
(S5) taking the input data {x|x₁, . . . , xₘ, . . . x_{M}} as an input of the DXN emission concentration prediction model; and calculating, successively by the RF-based DXN sub-model establishing module, the GBDT-based DXN sub-model establishing module and the simple average-based DXN integrated prediction module, a current DXN emission concentration value as a DXN emission concentration predicted value of the MSWI process.

2. The method of claim 1, wherein the training sample and input feature random sampling module is operated through steps of:
processing data of the process variable of the MSWI process by a Bootstrap method and a random subspace method (RSM);
extracting the training subset by the Bootstrap method, wherein the number of samples in the training subset is the same with the number of samples of the training sample set; and
introducing the RSM to randomly select some features to generate J training subsets comprising N training samples and M^{j} input features; expressed as follows:
$$\begin{matrix}X = {\{ {x}_{n} \}}_{n = 1}^{N} ∈ {R}^{N \times M} \\ y = {\{ {y}_{n} \}}_{n = 1}^{N} \\ J\end{matrix} \} ⇒ ⇒ \{ \begin{matrix}\{ {X}^{1} , {y}^{1} \} = {\{ {( {x}^{1 , {M}^{1}} , {y}^{1} )}_{n} \}}_{n = 1}^{N} \\ … \\ \{ {X}^{j} , {y}^{j} \} = {\{ {( {x}^{j , {M}^{j}} , {y}^{j} )}_{n} \}}_{n = 1}^{N} \\ … \\ \{ {X}^{J} , {y}^{J} \} = {\{ {( {x}^{J , {M}^{J}} , {y}^{J} )}_{n} \}}_{n = 1}^{N}\end{matrix} ; \qquad ( 1 )$$
wherein {X^{j}, y^{j}} is a jth training subset; (x^{j,M}^{^{j}}, y^{j}) is a nth input-output sample pair of the jth training subset; m=1,, . . . , M^{j}, M^{j} is the number of input features in the jth training subset; and M^{j}<<M.

3. The method of claim 2, wherein the RF-based DXN sub-model establishing module is operated through the following steps with the j^{th} training subset {(x^{j,M}^{^{j}}, y^{j})ₙ}ₙ₌₁^{N} as an example:
removing a duplicate sample from the jth training subset {(x^{j,M}^{^{j}}, y^{j})ₙ}ₙ₌₁^{N} caused by random sampling;
marking the duplicate sample as
$${\{ {( {x}^{j , {M}^{j}} , {y}^{j} )}_{{n}_{sel}} \}}_{{n}_{sel} = 1}^{{N}_{sel}} ;$$
splitting an input feature space into two areas, respectively R₁ and R₂ by taking a mth input feature x^{j,m} as a splitting variable and a value
$${x}_{{n}_{sel}}^{j , m}$$
corresponding to a nₛₑₗth sample as a splitting point:
$$\{ \begin{matrix}{R}_{1} ( m , {x}_{{n}_{sel}}^{j , m} ) = \{ {x}^{j , {M}^{j}} \vert {x}^{j , m} \leq {x}_{{n}_{sel}}^{j , m} \} \\ {R}_{2} ( m , {x}_{{n}_{sel}}^{j , m} ) = \{ {x}^{j , {M}^{j}} \vert {x}^{j , m} > {x}_{{n}_{sel}}^{j , m} \}\end{matrix} ; \qquad ( 2 )$$
finding a number of an optimal splitting variable and a value of the splitting point based on the following criterion by traversing all input features:
$${min}_{m , {x}_{{n}_{sel}}^{j , m}} {[ {min}_{{C}_{1}} {\sum }_{{x}^{j , m} ∈ {R}_{1} ( m , {x}_{{n}_{sel}}^{j , m} )} ( {y}_{1}^{j} - {C}_{1} ) )}^{2} + {min}_{{C}_{2}} {\sum }_{{x}^{j , m} ∈ {R}_{2} ( m , {x}_{{n}_{sel}}^{j , m} )} {( {y}_{2}^{j} - {C}_{2} )}^{2} ] ; \qquad ( 3 )$$
wherein y₁^{j} and y₂^{j} are a measured value of DXN emission concentration of the jth training subset in the R₁ and the R₂, respectively; and C₁ and C₂ are a mean value of a measured value of DXN emission concentration in the R₁ and the R₂, respectively;
repeating the above processes respectively for R₁ and R₂ until the number of training samples in a leaf node is less than a preset threshold θ_{RF} to split the input feature space into K areas; and marking the K areas as R₁, . . . , Rₖ, . . . , R_{K}, respectively; wherein K indicates the number of the leaf node of a classification and regression tree (CART);
the RF-based DXN sub-model established by the CART is expressed as follows:
$$\begin{matrix}{\hat{y}}_{R F}^{j} = {f}_{R F}^{j} ( \cdot ) = {\sum }_{k = 1}^{K} {c}_{R F}^{k} I ( {x}^{j , {M}^{j}} ∈ {R}_{k} ) ; & ( 4 ) \\ wherein {c}_{R F}^{k} = \frac{1}{{N}_{{R}_{k}}} {\sum }_{{n}_{{R}_{k}} = 1}^{{N}_{{R}_{k}}} {y}_{{n}_{{R}_{k}}}^{j} , {N}_{{R}_{k}} \leq {θ}_{R F} , & ( 5 ) ;\end{matrix}$$
wherein N_{R}_{ₖ} is the number of training samples in the area Rₖ;
$${y}_{{n}_{{R}_{k}}}^{j}$$
is a n_{R}_{ₖ} th measured value of DXN emission concentration of the jth training subset in the area Rₖ; I(⋅) is an indicator function; and when x^{j,M}^{^{j}}∉Rₖ, I(⋅)=1, otherwise I(⋅)=0;
a prediction error of the RF-based DXN sub-model established based on the jth training subset {(x^{j,M}^{^{j}}, y^{j})ₙ}ₙ₌₁^{N} is expressed as follows:
$${e}^{j , 0} = {y}^{j} - {\hat{y}}_{R F}^{j} = {\{ {( {e}^{j , 0} )}_{n} \}}_{n = 1}^{N} ; \qquad ( 6 )$$
wherein (e^{j,0})ₙ is a prediction error of DXN emission concentration based on a nth training sample; and
repeating the above processes to obtain J RF-based DXN sub-models {f_{RF}^{j}(⋅)}ⱼ₌₁^{J} established by the CART; and subtracting a predicted output {ŷ_{RF}^{j}}ⱼ₌₁^{J} of the J RF-based DXN sub-models respectively from a measured value fy to obtain the prediction error {e^{j,0}}ⱼ₌₁^{J}.

4. The method of claim 3, wherein the GBDT-based DXN sub-model establishing module is operated through steps of:
establishing multiple weak learner models “in series”; wherein an input data of a training subset of the multiple weak learner models is unchanged; a true value of output data of a training subset of a first GBDT-based DXN sub-model is an error between the predicted output of the RF-based DXN sub-model and the measured value; and a true value of output data of a training subset of other GBDT-based DXN sub-models is a prediction error of the GBDT-based DXN sub-model iterated in a previous iteration;
taking establishment of a jth GBDT-based DXN sub-model as an example, and supposing that there are I GBDT-based DXN sub-models to be established by the CART:
establishing a first GBDT-based DXN sub-model:
$${\hat{y}}_{GBDT}^{j , 1} = {{f}_{GBDT}^{j , 1} ( {\{ {x}^{j , {M}^{j}} )}_{n} \}}_{n = 1}^{N} , {\{ {( {e}^{j , 0} )}_{n} \}}_{n = 1}^{N} ) ) ; \qquad ( 7 )$$
wherein ŷ_{GBDT}^{j,1} is a predicted output of the first GBDT-based DXN sub-model;
a loss function of the first GBDT-based DXN sub-model is defined as follows:
$${L}_{G B D T} ( {y}^{j} , {\hat{y}}_{G B D T}^{j , 1} ) = \frac{1}{2} {\sum }_{n = 1}^{N} {( {( {e}^{j , 0} )}_{n} - {( {\hat{y}}_{G B D T}^{j , 1} )}_{n} )}^{2} ; \qquad ( 8 )$$
wherein (ŷ_{GBDT}^{j,1})ₙ is a predicted value of a nth sample in a jth training subset;
calculating an output residual e^{j,1} of the first GBDT-based DXN sub-model f_{GBDT}^{j,1}(⋅) expressed as follows:
$$\begin{matrix}{e}^{j , 1} = {e}^{j , 0} - {f}_{G B D T}^{j , 1} ( \cdot ) \\ = {y}^{i} - {f}_{R F}^{j} ( \cdot ) - {f}_{G B D T}^{j , 1} ( \cdot ) \\ = {y}^{j} - {\hat{y}}_{R F}^{j} - {\hat{y}}_{G B D T}^{j , 1}\end{matrix} ; \qquad ( 9 )$$
taking the e^{j,1} as a true value of output data of a training subset of a second GBDT-based DXN sub-model f_{GBDT}^{j,2}(⋅); wherein the second GBDT-based DXN sub-model is expressed as follows:
$${\hat{y}}_{G B D T}^{j , 2} = {f}_{G B D T}^{j , 2} ( {\{ {( {x}^{j , {M}^{j}} )}_{n} \}}_{n = 1}^{N} , {\{ {( {e}^{j , 1} )}_{n} \}}_{n = 1}^{N} ) ) ; \qquad ( 10 )$$
wherein (e^{j,1})ₙ is a prediction error of the first GBDT-based DXN sub-model for the nth sample;
repeating the above process; marking a ith (i≤I) GBDT-based DXN sub model as f_{GBDT}^{j,i}(⋅), wherein an output residual of the ith GBDT-based DXN sub-model is expressed as follows:
$$\begin{matrix}{e}^{j , i} = {y}^{j} - {f}_{R F}^{j} ( \cdot ) - {f}_{G B D T}^{j , 1} ( \cdot ) - , … , - {f}_{G B D T}^{j , i} ( \cdot ) \\ = {y}^{j} - {\hat{y}}_{R F}^{j} - {\hat{y}}_{G B D T}^{j , 1} - , … , - {\hat{y}}_{G B D T}^{j , i}\end{matrix} ; \qquad ( 11 )$$
after I−1 iterations, a true value of output data of a training subset of a Ith GBDT-based DXN sub-model is expressed as follows:
$${e}^{j , I - 1} = {y}^{j} - {\hat{y}}_{R F}^{j} - {\hat{y}}_{G B D T}^{j , 1} - , … , - {\hat{y}}_{G B D T}^{j , i} , … , - {y}_{G B D T}^{j , I - 1} ; \qquad ( 12 )$$
wherein ŷ_{GBDT}^{j,I-1} is a predicted output of a (I−1)th GBDT-based DXN sub-model; and
the Ith GBDT-based DXN sub-model is expressed as follows:
$${\hat{y}}_{G B D T}^{j , I} = {f}_{G B D T}^{j , I} ( {\{ {( {x}^{j , {M}^{j}} )}_{n} \}}_{n = 1}^{N} , {\{ {( {e}^{j , I - 1} )}_{n} \}}_{n = 1}^{N} ) ) ; \qquad ( 13 )$$
wherein (e^{j,I-1})ₙ is a prediction error of the (I−1)th GBDT-based DXN sub-model for the nth sample; and
the I GBDT-based DXN sub-models constructed based on the jth training subset are expressed as {f_{GBDT}^{j,i}(⋅)}ᵢ₌₁^{I}, and an output of the I GBDT-based DXN sub-models is expressed as {ê_{GBDT}^{j,i}}ᵢ₌₁^{I}.

5. The method of claim 4, wherein the simple average-based DXN integrated prediction module is operated through steps of:
indicating J RF-based DXN sub-models established in parallel as {f_{RF}^{j}(⋅)}ⱼ₌₁^{J}; and indicating J×I GBDT-based DXN sub-models established in series and parallel simultaneously as
$${\{ {( {f}_{G B D T}^{j , i} ( \cdot ) )}_{i = 1}^{I} \}}_{j = 1}^{J} ;$$
establishing one RF-based DXN sub-model and I GBDT-based DXN sub-models in parallel for the jth training subset; and taking a sum of a predicted output of the one RF-based DXN sub-model and the I GBDT-based DXN sub-models as a total output of the jth training subset, expressed as follows:
$$\begin{matrix}{\hat{y}}^{j} = {\hat{y}}_{R F}^{j} + {\hat{y}}_{G B D T}^{j , 1} + , … , + {\hat{y}}_{G B D T}^{j , i} , … , + {\hat{y}}_{G B D T}^{j , I - 1} \\ = {\hat{y}}_{R F}^{j} + {\sum }_{i = 1}^{I} {\hat{y}}_{G B D T}^{j , i} \\ = {f}_{R F}^{j} ( \cdot ) + {\sum }_{i = 1}^{I} {f}_{G B D T}^{j , i} ( \cdot )\end{matrix} ; and \qquad ( 14 )$$
since the J training subsets are parallel, combining the one RF-based DXN sub-model with the I GBDT-based DXN sub-models through a simple average weighting method; wherein the prediction model f_{DXN}(⋅)is expressed as follows:
$$\hat{y} = {f}_{DXN} ( \cdot ) = \frac{1}{J} {\sum }_{j = 1}^{J} {\hat{y}}^{j} = \frac{1}{J} {\sum }_{j = 1}^{J} ( {f}_{R F}^{j} ( \cdot ) + {\sum }_{i = 1}^{I} {f}_{G B D T}^{j , i} ( \cdot ) ) . \qquad ( 15 )$$
